(19) United States
(12) Patent Application Publication
(10) Pub. No.: US 2017/0112387 A1
(43) Pub. Date: Apr. 27, 2017

(54) BODY TEMPERATURE DETECTION AND CONTROL

(71) Applicant: Shreya Venkatesh, Mumbai (IN)

(72) Inventor: Shreya Venkatesh, Mumbai (IN)

(21) Appl. No.: 15/287,204

(22) Filed: Oct. 6, 2016

Related U.S. Application Data
(60) Provisional application No. 62/246,280, filed on Oct. 26, 2015.

(51) Int. Cl. A61B 5/01; G01K 1/02; A61F 7/10; A61F 7/00; A61F 7/08; G01K 13/00; A61B 5/00

(52) U.S. Cl. CPC A61B 5/01; G01K 13/002; G01K 1/024; A61B 5/0008; A61F 7/0097; A61F 7/08; A61F 7/10; A61F 2007/0096; A61F 2007/0054; A61F 2007/0056; A61B 2503/045

(57) ABSTRACT

A body temperature detection and control system, including a temperature sensing module configured to sense a body temperature, a processing module configured to determine whether the sensed body temperature is outside of a predetermined temperature range, and a response module configured to, if the sensed body temperature is determined to be outside of a predetermined temperature range, provide at least one of a notification and a remedial measure.

### TECHNICAL FIELD

The present disclosure relates to detection of body temperature, and in response to the body temperature being out of a predetermined temperature range, providing notification and/or enacting remedial measures.

### BACKGROUND

Thermoregulation refers to q physiological process of maintaining an internal body temperature within a normal range. Thermoregulation encompasses all processes and responses that balance heat production and heat loss to maintain body temperature within this normal range, which for humans is typically 37° C. regardless of the environmental temperature or activity level of the individual.

Thermoregulation is a negative feedback system, and the hypothalamus is an important temperature-regulating center. The hypothalamus has receptors that sense the temperature of blood flowing through the brain. In addition, temperature sensitive receptors in the skin send information to the hypothalamus about the temperature of the skin surface. In normal operating conditions, changes in core body temperature cause the hypothalamus to send nerve impulses to sweat glands, muscles, and blood vessels to raise or lower body temperature as appropriate.

In cold conditions, several mechanisms conserve and/or generate body heat. For instance, sweat production decreases, arrector pili muscles contract (lifting hair follicles upright and forming an insulating layer that traps heat), arterioles carrying blood to sub-surface skin capillaries constrict (routing blood away from cooler skin surface into the warmer body core). In addition, muscle shivering that can produce heat as respiration is an exothermic reaction in muscle cells. And mitochondria can convert fat (especially brown fat abundant in newborn babies) directly into heat, increasing body temperature of all cells in the body.

Preterm or low-birth weight babies are more vulnerable to body heat loss than adults for a variety of reasons. For instance, behavioral responses to environmental temperature changes are almost non-existent in newborns. And preterm babies have limited ability to mobilize energy sources to combat high heat losses, particularly from evaporation. Such babies may incur body heat loss via radiation, convection, conduction, and evaporation. If body heat loss is not effectively preempted, neonates will be subjected to the effects of cold stress or hypothermia which can lead to death.

### BRIEF DESCRIPTION OF THE DRAWINGS

FIG. 1 is a schematic diagram illustrating a body temperature detection and control system in accordance with an embodiment of the disclosure.

FIG. 2 is a perspective diagram illustrating the processing module and temperature sensing module of FIG. 1.

FIG. 3 illustrates a flowchart of body temperature detection and control method in accordance with an embodiment of the disclosure.

### DETAILED DESCRIPTION

Newborn babies, also referred to as neonates, should be homoeothermic, but their ability to stay warm may be readily overwhelmed by environmental factors. Newborn babies can lose heat by, e.g., evaporation soon after birth (due to evaporation of amniotic fluid from skin surface), conduction (by coming in contact with cold objects), convection (by air currents in which cold air replaces warm air around baby), and radiation (to colder solid objects in vicinity). A newborn baby is more prone to develop hypothermia because of their large surface area per module of body weight. A low birth weight baby has decreased thermal insulation due to less subcutaneous fat and reduced amount of brown fat, which respectively lessen temperature insulation and non-shivering heat production.

As used herein, the terms “newborn,” “newborn baby,” “neonate,” refer to mammalian babies about six weeks old or less and also to mammalian babies less than six months old but, due to premature birth or illness or condition, have a body weight approximately that of a normal and similar in kind mammalian baby of less than six weeks old.

Neonatal hypothermia often occurs as a result of a lack of attention by healthcare providers, and continues to be an important cause of neonatal deaths. Hypothermia has been defined by World Health Organization as body temperature below the normal range (36.5° C.-37.5° C.) and has been sub-classified into three grades: mild (36.0° C.-36.5° C.), moderate (32.0° C.-35.9° C.), and severe (<32.0° C.) hypothermia.

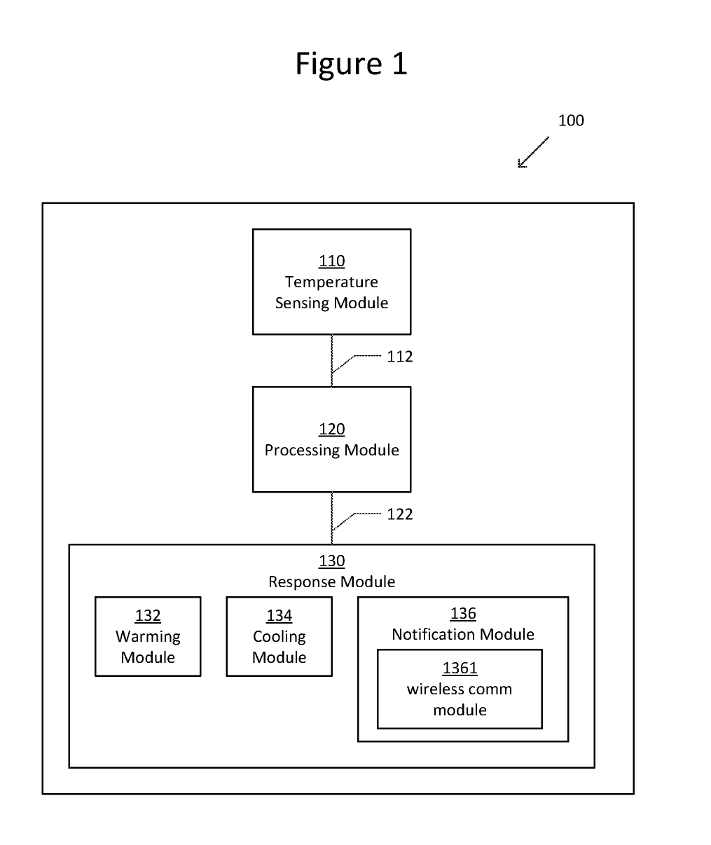

FIG. 1 is a schematic diagram illustrating a body temperature detection and control system 100 in accordance with an embodiment of the disclosure. The system 100 inhibits a body temperature of a newborn baby or other living being from fluctuating outside of a predetermined temperature range that is considered to be a normal body temperature range.

The system 100 comprises a temperature sensing module 110, a processing module 120, and a response module 130. The temperature sensing module 110 is coupled to the processing module 120 by a first communication line 112. The processing module is coupled to the response module 130 by a second communication line 122. Each of the first and second communication lines 112, 122 may be any of a fiber optic cable, an electrical cable, a wireless link, a combination thereof, or any other communication line as suitable for the intended purpose.

The temperature sensing module 110 is configured to sense a body temperature of a newborn baby or other living being. The temperature sensing module 110 comprises a temperature sensor (not shown), which may be an electronic thermometer, an infrared thermometer, a resistance temperature detector, a thermocouple, a thermistor, an e-health platform temperature sensor, a combination thereof, or any other temperature sensor as suitable for the intended purpose.

The temperature sensing module 110 may comprise a thermistor based thermometer configured for positioning and operation on a newborn's or other living being's body. When a main circuit (described below) is coupled to a power source, such as a battery or a power outlet, the main circuit signals the thermistor instructions to begin recording body temperature. The working of the thermistor can be programmed using the Steinhart Hart Equation which converts resistance values to concrete temperature values. Such temperature values may be stored in a memory of the main circuit and transmitted to the processing module 120.

The temperature sensing module 110 may sense the body temperature continuously and communicate the sensed body temperature to the processing module 120. Alternatively, the temperature sensing module 110 may sense the body temperature continuously and communicate the body temperature to the processing module 120 at predetermined intervals. Alternatively, the temperature sensing module 110 may communicate the body temperature to the processing module 120 after the predetermined intervals. Alternatively, the temperature sensing module 110 may communicate the body temperature to the processing module 120 when the body temperature is determined to be outside of the predetermined temperature range.

The processing module 120 is configured to determine whether the sensed body temperature from the temperature sensing module 110 is outside of a predetermined temperature range. The predetermined range is generally a normal body temperature range for a newborn baby or other human being, or any other range as suitable for the intended purpose.

In some embodiments, the processing module 120 is configured to process the body temperature data into an output indicative of an abnormal body temperature that is low when the body temperature data indicates that the body temperature of a newborn baby is at least 1° C., 2° C., or 3° C. less than a predetermine normal body temperature value for the newborn baby (e.g., 36.5° C.-37.5° C.). In some embodiments, the processing module 120 is configured to process the body temperature data into an output indicative of an abnormal body temperature that is high when the body temperature data indicates that the body temperature of the newborn baby is at least 1° C., 2° C., or 3° C. greater than a predetermine normal body temperature value for the newborn baby (e.g., 36.5° C.-37.5° C.).

The processing module 120 may comprise a memory and processing module in the main circuit. The processing module 120 is programmed to sends alert signals to the response module 130 when a sensed temperature is outside the predetermined normal temperature range.

The response module 130 comprises any one or more of a warming module 132, a cooling module 134 and a notification module 136. The response module 130 is, if the sensed body temperature is outside of the predetermined temperature range, configured to provide at least one of a notification by the notification module and a remedial measure by either the warming module 132 or the cooling module 134.

The warming module 132 is configured to provide heat when the processing module 120 determines that the sensed body temperature is less than the predetermined temperature range, and may optionally continue to provide heat until the sensed body temperature is within the predetermined temperature range. The warming module 132 may be any of an electric heat pad, a liquid heat pad, an electric warming blanket, a liquid warming blanket, a heat light, a combination thereof, or any other warming device as suitable for the intended purpose.

The cooling module 134 is configured to provide heat reduction when the processing module 120 determines that the sensed body temperature is greater than the predetermined temperature range, and may optionally continue to provide heat reduction until the sensed body temperature is within the predetermined temperature range. The cooling module 134 may be a liquid cooling pad, a liquid cooling blanket, a fan, an air conditioning module, a combination thereof, or any other cooling module as suitable for the intended purpose.

The warming module 132 or the cooling module 134 may be switched on at or around the time the processing module 120 sends an abnormal body temperature notification to a healthcare provider. In such embodiments, the warming module 132 or the cooling module 134 would operate to maintain the newborn's or other living being's body temperature until the notified healthcare provider attends to the neonate or other living being. In some embodiments, the warming module 132 or cooling module 134 comprises an external relay configured to cease operation at a threshold body temperature in order to avoid overheating or under heating the newborn's or other living being's body. Such embodiments ensure the safety of the newborn or living being.

The notification module 136 is configured to issue a notification. This notification may be an email, a text message, an alarm, a prerecorded phone call, a combination thereof, or any other notification as suitable forth intended purpose. The notification module 136 may be configured to repeat or continue the notification until the notification is deactivated, such as by a health care worker. Alternatively, the notification module 136 may be configured to repeat or continue the notification the sensed body temperature returns to be within the predetermined normal temperature range.

Healthcare providers referred to herein may include, without limitation, doctors, nurses, midwives, babysitters, family members involved with ensuring and/or monitoring the health and/or wellness of a newborn baby or other living being.

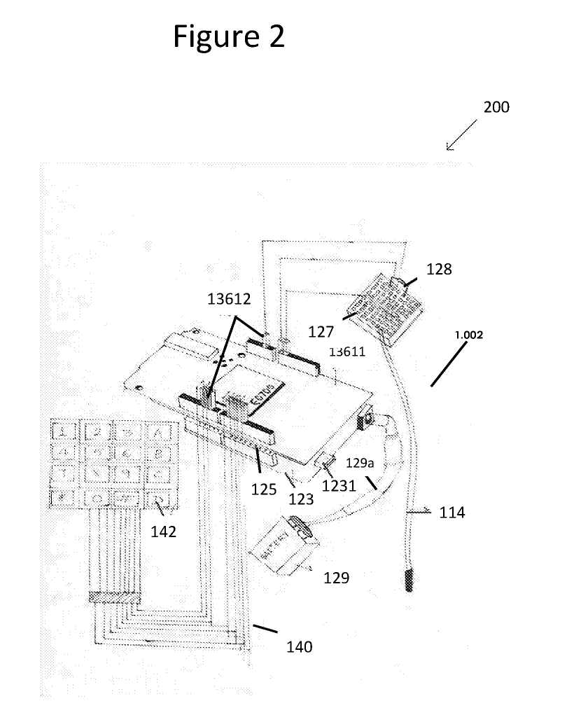

The notification module 136 comprises a wireless communication module 1361 and a mobile application module 1362. The wireless communication module 1361 is coupled to the mobile application module 1362 h a wireless shield (shown in FIG. 2 and described below). The wireless communication module 1361 may be a global system for mobile communications (GSM) module, an Ethernet module, a wifi module or a combination thereof. The wireless communication module 1361 and the mobile application module 1362 operate together to notify a healthcare provider upon receipt of an indication that the temperature of a neonate or other living being drops below or moves above the normal range by means of a sending a Short Message Service (SMS or text message). The SMS notification content can differ in order to convey variations in the degree of temperature abnormality detected by the temperature sensing module 110 and transmitted to the notification module 136. For example, the SMS notification content can be categorized based on the severity of hypothermia (e.g., mild, moderate, or severe) detected by a temperature sensing module and transmitted to the notification module 136. Messages can also be sent using wifi and/or Ethernet modules. The mobile application module 1362 may be configured to store historical temperature readings and notify the health care provider by means of a push notification generated by the mobile application module 1362 when the temperature becomes outside the predetermined normal temperature range.

FIG. 2 is a perspective diagram 200 illustrating the processing module 120 and temperature sensing module 110 of FIG. 1. The details of the processing module 120 and the temperature sensing module 110 shown in FIG. 2 are merely examples. Those of ordinary would understand that the specific elements shown could be substituted with other elements suitable for the intended purposes.

The processing module 120 comprises a main circuit 123, which may be an Arduino Uno, for example. The main circuit 123 comprises a USB cable port 1231 that couples the main circuit 123 to a computer using a USB cable to input programs, a power adapter port in order to couple the main circuit 123 to an external power source, connector pins 125 that enable other shields or circuits to be mounted to the main circuit 123 and act as a medium of communication between the circuits.

A GSM shield 124, such as an Arduino GSM shield, comprises a USB cable port (not shown) that couples the GSM shield 124 to a computer to be able to input programs into the GSM shield 124 for use as a separate module. The GSM shield 124 may also comprise a sim card holder (not shown) to place a sim card for communication purposes, and sockets (not shown) for the jumper cables 126.

The power source may comprise an alkaline battery 129 coupled to the main circuit 123 by a power cord 129a.

A user input module 140 comprises a keypad 142 for inputting a mobile number and/or any other data into the device 100. The keypad 142 is coupled to the GSM shield 124 via jumper cables 126.

The temperature-sensing module 110 may comprise an epoxy thermistor 114, or other suitable temperature sensing device, coupled to the GSM shield 124 via the jumper cables 126 and forming an external circuit on a breadboard 127 having a 10 kΩ resistor 128.

When the processing module 120 receives a temperature value that is outside of the predetermined temperature range, the processing module 120 may send two separate signals. The first signal may be transmitted to the notification module 136. The second signal may be sent to turn on the warming module 132 or cooling module 134.

The wireless communication module 1361 comprises the GSM shield 124 mounted on the main circuit 123, and there may be a keypad 142 coupled to the GSM shield 214. The GSM shield 124 may use a sim card for sending SMS. The wireless communication module 1361 could also or alternatively consist of a wifi shield which could connect to any mobile application wirelessly. The notification module 136 (wireless communication module 1361 in combination with the mobile application module 1362) upon receiving signals from the processing module 120 sends a message, which may be pre-written programmed, to a remote mobile or other communication device in the form of an SMS message, voice mail, or other suitable message form. The mobile number or communication identification device that is entered using the keypad 142 or by some other input means is stored in the program memory and is called when the notification module 136 is triggered. The SMS messages are programmed to provide the healthcare provider with the present condition of the newborn or other living being monitored. The wireless communication module 1361 may use a wifi shield or any wireless shield or circuit to couple wirelessly to the mobile application module 1362. In such cases, the temperature values are sent along with alert notifications to the mobile application module 1362, which stores these values and sends a push notification when the temperature is outside of the predetermined normal temperature range.

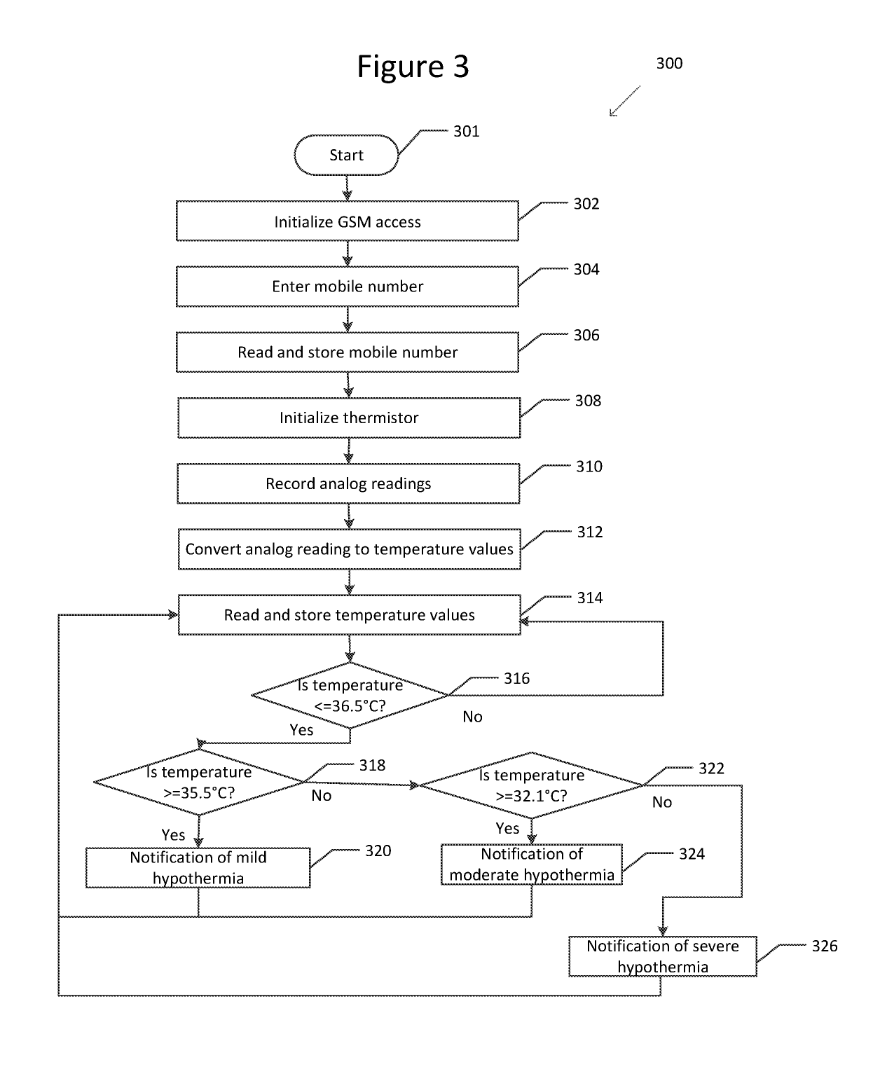

FIG. 3 illustrates a flowchart of body temperature detection and control method 300 in accordance with an embodiment of the disclosure

On turning on the device 100 by connecting it with a battery 129 or other power source (Start 301), GSM access to the device 100 is initialized when the sim card in the GSM shield 124 connects to the device 100 (Step 302). A mobile number entered using the keypad 142 (Step 304) is stored in a memory (Step 306). On entering the mobile number, the thermistor 114 is initialized (Step 308) and senses analog temperature readings (Step 310). These analog readings are converted into temperature values using the Steinhart Hart equation (Step 312), and these temperature values are stored in a memory (Step 314). Each time a temperature value is stored in the memory, the temperature value is compared with a threshold value for hypothermia, that is, the threshold value is less than or equal to 36.5° Celsius (Step 316). If this condition is false, the program returns to reading temperature values (Step 316). However, if this condition is true, the severity of the hypothermia is determined. More specifically, if it is determined if the temperature is less than or equal to 35.5° Celsius (Step 318), the GSM shield 124 is triggered, a notification of mild hypothermia is issued (Step 320), and then temperatures continue to be recorded (Step 314). However, if the condition is false, it is determined if the temperature is greater than than or equal to 32.1° Celsius (Step 322). If this final condition is true, a notification of moderate hypothermia is issued (Step 324), and then temperatures continue to be recorded (Step 314). Otherwise, a notification of severe hypothermia is issued (Step 326), and then temperatures continue to be recorded (Step 314).

The method illustrated by the flowchart of FIG. 3 is merely an example. Modification may be made without departing from the spirit and scope of the disclosure. For example, rather than testing for hypothermia, the method may alternatively test for hyperthermia. Also, the notification may be an email, a text message, an alarm, a prerecorded phone call, a combination thereof, or any other notification as suitable forth intended purpose. The notification may repeat or continue until the notification is deactivated, such as by a health care worker. Alternatively, the notification may repeat or continue until the sensed body temperature returns to be within the predetermined temperature range.

Although the disclosure has been provided in the context of certain embodiments described and illustrated as comprising modules and components arranged in particular ways, it will be understood by those skilled in the art that the disclosure extends beyond the specifically described and illustrated embodiments to other alternative embodiments having alternative equivalent components and/or arrangements of components, and/or uses and obvious modifications and equivalents thereof. Accordingly, the disclosure is not intended to be limited by the specific disclosures of embodiments herein.

What is claimed is:

1. A body temperature detection and control system, comprising:
a temperature sensing module configured to sense a body temperature;
a processing module configured to determine whether the sensed body temperature is outside of a predetermined temperature range; and
a response module configured to, if the sensed body temperature is determined to be outside of the predetermined temperature range, provide at least one of a notification and a remedial measure.

2. The system of claim 1, wherein the temperature sensing module comprises a temperature sensor selected from the group of temperature sensors consisting of an electronic thermometer, an infrared thermometer, a resistance temperature detector, a thermocouple, a thermistor, an e-health platform temperature sensor, and a combination thereof.

3. The system of claim 1, further comprising a first communication line coupling the temperature sensing module and the processing module, and a second communication line coupling the processing module and the response module.

4. The system of claim 3, wherein each of the first and second communication lines is selected from the group of communication lines consisting of a fiber optic cable, an electrical cable, a wireless link, and a combination thereof.

5. The system of claim 1, wherein the temperature sensing module is further configured to sense the body temperature continuously and communicate the body temperature to the processing module continuously.

6. The system of claim 1, wherein the temperature sensing module is further configured to sense the body temperature continuously and communicate the body temperature to the processing module at predetermined intervals.

7. The system of claim 1, wherein the temperature sensing module is further configured to sense the body temperature continuously and communicate the body temperature to the processing module when the body temperature is determined to be outside of the predetermined temperature range.

8. The system of claim 1, wherein the processing module is further configured to determine that the sensed body temperature is outside of the predetermined temperature range when the sensed body temperature is at least 1° C. greater than a predetermined normal body temperature.

9. The system of claim 1, wherein the processing module is further configured to determine that the sensed body temperature is outside of the predetermined temperature range when the sensed body temperature is at least 1° C. less than a predetermined normal body temperature.

10. The system of claim 1, wherein the response module comprises a warming module configured to provide heat when the processing module determines that the sensed body temperature is less than the predetermined temperature range

11. The system of claim 11, wherein the warming module is configured to provide heat until the sensed body temperature is within the predetermined temperature range.

12. The system of claim 10, wherein the warming module is selected from the group of warming modules consisting of an electric heat pad, a liquid heat pad, an electric warming blanket, a liquid warming blanket, a heat light, and a combination thereof.

13. The system of claim 1, wherein the response module comprises a cooling module configured to provide heat reduction when the processing module determines that the sensed body temperature is greater than the predetermined temperature range.

14. The system of claim 13, wherein the cooling module is configured to provide heat reduction and until the sensed body temperature is within the predetermined temperature range.

15. The system of claim 13, wherein the cooling module is selected from the group of cooling modules consisting of a liquid cooling pad, a liquid cooling blanket, a fan, an air conditioning module, and a combination thereof.

16. The system of claim 1, wherein the response module comprises a notification module configured to issue a notification, wherein the notification is at least one of an email, text message, alarm and prerecorded phone call.

17. The system of claim 16, wherein the notification repeats or continues until the notification is deactivated.

18. The system of claim 16, wherein the notification repeats or continues until the sensed body temperature is within the predetermined temperature range.

17. The system of claim 1, wherein the body temperature is a body temperature of a newborn baby.

18. The system of claim 1, wherein the body temperature is a body temperature of a living being.
